(19) United States
(12) Patent Application Publication
(10) Pub. No.: US 2025/0143738 A1
(43) Pub. Date: May 8, 2025

(54) DEVICES, SYSTEMS, AND METHODS FOR TUNNELING BETWEEN TISSUE LAYERS

(71) Applicant: BOSTON SCIENTIFIC SCIMED, INC., MAPLE GROVE, MN (US)

(72) Inventors: Austin Grant Johnson, Worcester, MA (US); Paul Smith, Smithfield, RI (US); Ryan V. Wales, Northborough, MA (US)

(73) Assignee: BOSTON SCIENTIFIC SCIMED, INC., MAPLE GROVE, MN (US)

(21) Appl. No.: 18/935,123

(22) Filed: Nov. 1, 2024

Related U.S. Application Data
(60) Provisional application No. 63/547,013, filed on Nov. 2, 2023.

(51) Int. Cl. A61B 17/32; A61B 17/00

(52) U.S. Cl. CPC A61B 17/32; A61B 17/00234; A61B 2017/00296; A61B 2017/00477; A61B 2017/320048

(57) ABSTRACT

Blunt tissue dissection using a sharp cutting instrument to make an initial cut in tissue and an inflatable device to further separate tissue layers on either side of the cut. The sharp cutting instrument may be advanced through the lumen of an elongate delivery member such as through a working channel of a medical scope. The inflatable device is inserted over the exterior of the elongate delivery member.

### CROSS-REFERENCE TO RELATED APPLICATIONS

This application claims the benefit of priority under 35 U.S.C. § 119 to U.S. Provisional Application No. 63/547,013, filed Nov. 2, 2023, the entire disclosure of which is hereby incorporated by reference herein for all purposes.

### FIELD

The present disclosure relates generally to the field of medical devices for accessing spaces within anatomical structures. In particular, the present disclosure relates to medical devices, systems, and methods for cutting or tunneling in and/or between tissue layers of an anatomical structure.

### BACKGROUND

Various devices, assemblies, systems, methods, techniques, etc., exist for cutting, dissecting, resecting, excising, etc., anatomical structures (e.g., biological tissue, including lesions, etc.). The configurations of the cutting device (which may include a blade, needle, laser, or other structure capable of cutting anatomical structures) may be selected based on the particular procedure to be performed. The procedures may be performed using open surgery (accessing the interior of a patient's body by cutting open the body) or minimally invasive surgery (e.g., percutaneously, laparoscopically, endoscopically, etc.). One endoscopic procedure is third space endoscopy (also known as submucosal endoscopy), which may access deeper layers of tissue within the body (e.g., the gastrointestinal (GI) tract) by tunneling into the tissue, such as between structurally differentiated layers of tissue. For instance, in the GI system, tunneling may be performed in the submucosal space with an endoscope, without compromising the integrity of the overlying mucosa. Typically, a fluid (e.g., water, saline, air, etc.) is injected at the treatment site to elevate/lift the tissue (to create a “bleb”) to facilitate cutting of the tissue, such as with a knife. For instance, a lifting agent may be injected into the submucosal layer to separate the mucosal layer from the muscularis layer. A sharp cutting instrument such as a blade, a knife, an electrosurgical knife, etc., may then be used to cut through the submucosa. While tunneling, the medical scope (e.g., gastroscope) may have a tapered distal cap mounted over the distal end thereof to help pass the scope through the tissue at the target site. Blunt tissue dissection may be performed using a mechanical force to separate tissue layers. In some aspects, a scope cap may provide a form of blunt tissue dissection as the scope tapers through the tissue. However, cutting instruments, such as electrosurgical knives, typically are predominantly used to tunnel through tissue in third space endoscopic procedures. Typically, the cutting instrument is used to make multiple small cuts, typically sequentially, to create a desired separation of tissue gradually, such as to reduce the risk of creating too large a cut in the tissue. Safer and more efficient tunneling devices, systems, methods, etc., would be welcome in the field. It is with respect to these and other considerations that the present improvements may be useful.

### SUMMARY

This Summary is provided to introduce, in simplified form, a selection of concepts described in further detail below in the Detailed Description. This Summary is not intended to necessarily identify key features or essential features of the claimed subject matter, nor is it intended as an aid in determining the scope of the claimed subject matter. One of skill in the art will understand that each of the various aspects and features of the present disclosure may advantageously be used separately in some instances, or in combination with other aspects and features of the disclosure in other instances, whether or not described in this Summary. No limitation as to the scope of the claimed subject matter is intended by either the inclusion or non-inclusion of elements, components, or the like in this Summary.

In accordance with various principles of the present disclosure, a system for tunneling between tissue layers includes an elongate delivery member; a cutting device deliverable with the elongate delivery member; and an inflatable device positionable on a distal end of the elongate delivery member, wherein the inflatable device is shaped to facilitate insertion into a cut area of tissue cut by the cutting device.

In some aspects, the elongate delivery member is tubular and defines a delivery lumen therethrough. In some aspects, the cutting device is deliverable through the delivery lumen of the elongate delivery member. In some aspects, the inflatable device is deliverable over the exterior of the elongate delivery member. In some aspects, the elongate delivery member is a medical scope, and the delivery lumen is the working channel of the medical scope.

In some aspects, the inflatable device is deliverable over the exterior of the elongate delivery member. In some aspects, the inflatable device is axially translatable distally and proximally over the exterior of the elongate delivery member. In some aspects, the inflatable device is mounted over a sheath and the sheath is axially translatable distally and proximally over the elongate delivery member. In some aspects, the inflatable device is movable distally off the exterior of the elongate delivery member.

Additionally or alternatively to the above features, the system may include an adaptor configured to maintain the inflatable device with respect to the elongate delivery member. In some aspects, the adaptor has a distal end tapered from a position adjacent a distal end of the inflatable device distally to the exterior of the elongate delivery member. In some aspects, the adaptor is formed of a compliant material capable of fitting over elongate delivery members having different outer diameters.

In some aspects, the inflatable device is tapered in a distal direction from an exterior thereof to an exterior of the elongate delivery member. In some aspects, the inflatable device is tapered in a proximal direction from an exterior thereof to an exterior of the elongate delivery member.

In some aspects, the cutting device is a sharp cutting device selected from the group consisting of: a blade, a knife, or an electrosurgical knife.

In accordance with various principles of the present disclosure, a method of performing blunt dissection of tissue includes advancing a cutting device to a target site within a patient; cutting tissue with the cutting device; advancing an inflatable device into the cut tissue; and inflating the inflatable device to separate the cut tissue further.

In some aspects, the method further includes advancing the cutting device into the mucosa of the patient to separate the mucosa from the submucosa.

In some aspects, the method further includes advancing the cutting device through a lumen of an elongate delivery member and advancing the inflatable member over the exterior of the elongate delivery member.

In accordance with various principles of the present disclosure, a method of performing endoscopic tissue dissection includes advancing a cutting device through a working channel of a medical scope to a target site within a patient; cutting tissue with the cutting device; advancing an inflatable device over the medical scope and into the cut tissue; and inflating the inflatable device to separate the cut tissue further.

In some aspects, the method further includes advancing the cutting device into the mucosa of the patient to separate the mucosa from the submucosa.

These and other features and advantages of the present disclosure, will be readily apparent from the following detailed description, the scope of the claimed invention being set out in the appended claims. While the following disclosure is presented in terms of aspects or embodiments, it should be appreciated that individual aspects can be claimed separately or in combination with aspects and features of that embodiment or any other embodiment.

### BRIEF DESCRIPTION OF THE DRAWINGS

Non-limiting embodiments of the present disclosure are described by way of example

with reference to the accompanying drawings, which are schematic and not intended to be drawn to scale. The accompanying drawings are provided for purposes of illustration only, and the dimensions, positions, order, and relative sizes reflected in the figures in the drawings may vary. For example, devices may be enlarged so that detail is discernable, but is intended to be scaled down in relation to, e.g., fit within a working channel of a delivery catheter or endoscope. For purposes of clarity and simplicity, not every element is labeled in every figure, nor is every element of each embodiment shown where illustration is not necessary to allow those of ordinary skill in the art to understand the disclosure.

The detailed description will be better understood in conjunction with the accompanying drawings, wherein like reference characters represent like elements, as follows:

FIG. 1 illustrates a schematic view of a tunneling system formed in accordance with various principles of the present disclosure being advanced to a treatment site.

FIG. 2A illustrates a schematic representation of a treatment site with a cutting device of a tunneling system formed in accordance with various principles of the present disclosure creating an initial cut.

FIG. 2B illustrates a schematic representation similar to that of FIG. 2A, but with the distal end of the tunneling system advanced further distally into the cut tissue.

FIG. 2C illustrates a schematic representation similar to that of FIG. 2B, but with the inflatable device of the tunneling system inflated to perform blunt dissection of the tissue cut by the cutting device.

FIG. 3 illustrates an elevational view of schematic representation of an example of an embodiment of a tunneling system formed in accordance with various principles of the present disclosure with an adaptor.

FIG. 4 illustrates a cross-sectional view along line IV-IV of FIG. 3.

FIG. 5A illustrates an elevational view of schematic representation of an example of an embodiment of a tunneling system formed in accordance with various principles of the present disclosure with an adaptor.

FIG. 5B illustrates an elevational view similar to that of FIG. 5A, but with the inflatable device proximally retracted relative to the position illustrated in FIG. 5A.

FIG. 6 illustrates a cross-sectional view along line VI-VI of FIG. 5A.

FIG. 7A illustrates a schematic representation of a tunneling system formed in accordance with various principles of the present disclosure being advanced to a treatment site.

FIG. 7B illustrates a schematic representation similar to that of FIG. 7A, but with the elongate delivery member proximally retracted and separated from the inflatable device.

### DETAILED DESCRIPTION

The following detailed description should be read with reference to the drawings, which depict illustrative embodiments. It is to be understood that the disclosure is not limited to the particular embodiments described, as such may vary. All apparatuses and systems and methods discussed herein are examples of apparatuses and/or systems and/or methods implemented in accordance with one or more principles of this disclosure. Each example of an embodiment is provided by way of explanation and is not the only way to implement these principles but are merely examples. Thus, references to elements or structures or features in the drawings must be appreciated as references to examples of embodiments of the disclosure, and should not be understood as limiting the disclosure to the specific elements, structures, or features illustrated.

Other examples of manners of implementing the disclosed principles will occur to a person of ordinary skill in the art upon reading this disclosure. In fact, it will be apparent to those skilled in the art that various modifications and variations can be made in the present disclosure without departing from the scope or spirit of the present subject matter. For instance, features illustrated or described as part of one embodiment can be used with another embodiment to yield a still further embodiment. Thus, it is intended that the present subject matter covers such modifications and variations as come within the scope of the appended claims and their equivalents.

It will be appreciated that the present disclosure is set forth in various levels of detail in this application. In certain instances, details that are not necessary for one of ordinary skill in the art to understand the disclosure, or that render other details difficult to perceive may have been omitted. The terminology used herein is for the purpose of describing particular embodiments only, and is not intended to be limiting beyond the scope of the appended claims. Unless defined otherwise, technical terms used herein are to be understood as commonly understood by one of ordinary skill in the art to which the disclosure belongs. All of the devices and/or methods disclosed and claimed herein can be made and executed without undue experimentation in light of the present disclosure.

As used herein, “proximal” refers to the direction or location closest to the user (medical professional or clinician or technician or operator or physician, etc., such terms being used interchangeably herein without intent to limit, and including automated controller systems or otherwise), etc., such as when using a device (e.g., introducing the device into a patient, or during implantation, positioning, or delivery), and/or closest to a delivery device, and “distal” refers to the direction or location furthest from the user, such as when using the device (e.g., introducing the device into a patient, or during implantation, positioning, or delivery), and/or closest to a delivery device. “Longitudinal” means extending along the longer or larger dimension of an element. A “longitudinal axis” extends along the longitudinal extent of an element, though is not necessarily straight and does not necessarily maintain a fixed configuration if the element flexes or bends, and “axial” generally refers to along the longitudinal axis. However, it will be appreciated that reference to axial or longitudinal movement with respect to the above-described systems or elements thereof need not be strictly limited to axial and/or longitudinal movements along a longitudinal axis or central axis of the referenced elements. “Central” means at least generally bisecting a center point and/or generally equidistant from a periphery or boundary, and a “central axis” means, with respect to an opening, a line that at least generally bisects a center point of the opening, extending longitudinally along the length of the opening when the opening comprises, for example, a tubular element, a channel, a cavity, or a bore. As used herein, a “lumen” or “channel” or “bore” or “passage” is not limited to a circular cross-section. As used herein, a “free end” of an element is a terminal end at which such element does not extend beyond. It will be appreciated that terms such as at or on or adjacent or along an end may be used interchangeably herein without intent to limit unless otherwise stated, and are intended to indicate a general relative spatial relation rather than a precisely limited location. Finally, reference to “at” a location or site is intended to include at and/or about the vicinity of (e.g., along, adjacent, proximate, etc.) such location or site. As understood herein, corresponding is intended to convey a relationship between components, parts, elements, etc., configured to interact with or to have another intended relationship with one another.

In accordance with various principles of the present disclosure, a device and system is configured to perform and/or facilitate performing “third space” procedures and/or procedures in other transluminal spaces of a patient which are generally difficult to access with current devices. Third space endoscopy, which is also known as submucosal endoscopy, involves tunneling through various layers of tissue (e.g., the various linings of the digestive system) to remove lesions that are deeper than the surface of the tissue, and/or to treat conditions that are deep within the tissue (e.g., in the muscle layer of the digestive system lining). Third space procedures involve accessed deep into tissue by tunneling into the tissue (e.g., in the submucosal space or beyond) without compromising the integrity of the overlying tissue (e.g., the mucosa). The “third space” of a subject may be a space inside a wall of a gastrointestinal (GI) tract of the subject, such as in or between a mucosal layer of tissue and a submucosal layer of tissue. Examples of third space procedures include, but are not limited to, per oral endoscopic myotomy (POEM), endoscopic submucosal dissection (ESD), Zenker's diverticulotomy (ZPOEM), endoscopic gastric pyloromyotomy (GPOEM), submucosal tunneling endoscopic resection (STER), and tunneling recanalization of the esophagus (POETRE), among others. These third space procedures and/or other suitable procedures may be used to treat esophageal sphincters, pyloric sphincters, subepithelial tumors, and/or other anatomical structures.

Some third space procedures (e.g., POEM, STER, POETRE) may include tunneling to or into the sub-mucosal tissue layer of the subject and performing one or more additional procedural steps. To create the tunnel, an incision may be made through the mucosa to extends from the mucosal layer of tissue to or into the submucosal layer of tissue to separate the mucosa from the muscularis layers of tissue such that a diameter of a medical scope may fit through the opening/cut. The creating of the incision from the mucosal tissue layer to or into the submucosal tissue layer can be challenging and time intensive. When tissue is cut, the tissue layers on either side of the cut may still remain close to each other.

In accordance with various principles of the present disclosure, an inflatable device is advanced into the cut region of tissue to put the connective tissue between the layers of tissue on either side of the cut into tension to facilitate further separation of the layers. Such use of an inflatable device in conjunction with a cutting device enables safer and/or less time intensive procedures such as third space endoscopy procedures. For instance, the cutting device may be used to initiate separation of tissue at a target site, and the inflatable device may then be used to separate the cut tissue. As may be appreciated, an inflatable device formed in accordance with various principles of the present disclosure may be inflated or deflated as needed to achieve the desired blunt tissue dissection. For instance, tissue may be initially cut with the cutting device of the system. Optionally, fluid may be injected into the target site to facilitate such cutting by creating a bleb at the target site prior to cutting. The inflatable device may be inserted within the cut and inflated to exert pressure (e.g., mechanical radial and/or normal forces against the tissue) causing the tissue to be pushed apart, such as may be achieved by a blunt instrument. More particularly, the inflatable device is advanced into the cut region to put the connective tissue between the layers of tissue on either side of the cut in tension to facilitate further separation of the layers. Even more particularly, when tissue is cut, the tissue layers on either side of the cut may still remain close to each other. For instance, a cut may be made through the mucosa to the submucosa to separate the mucosa from the muscularis layers of tissue. Upon separating the tissue with the inflatable device, the medical professional may observe the amount/extent of tissue which has been cut to determine if further cutting is medically indicated.

In some aspects, the inflatable device is positioned and/or configured to facilitate insertion into a cut region of tissue. For instance, a distal portion of the inflatable device may be tapered or wedge-shaped or otherwise narrower than more proximal portions to facilitate insertion into a cut tissue. Upon further insertion into the tissue, the widening of the inflatable device, such as a result of inflation of the device (e.g., if the inflatable device is initially inserted in an uninflated configuration) and/or the shape/dimensions/configuration of the inflatable device (with a distal portion narrower than a proximal portion in a direction across the cut and extending between the separated tissue layers), separates the cut tissue layers.

In some aspects of the present disclosure, the cutting device is delivered through a lumen of a tubular elongate delivery member of a treatment system, and the inflatable device is delivered over (e.g., around the exterior of) the tubular elongate delivery member. In some aspects, the elongate delivery member is a medical scope, and the cutting device is delivered through the working channel of the medical scope. In some aspects, the inflatable device is independently advanceable or retractable with respect to the elongate delivery member

In embodiments of a tunneling system with a medical scope as a elongate delivery member, the medical scope may have a visualization device (e.g., a camera, an optic fiber, etc.). The scope may be used to visualize the cut area, such as upon separation of the cut tissue by the inflatable device. The inflatable device may be advanced to a position adjacent the visualization device, and/or distal to the distalmost end of the elongate delivery member (and thus distal to the visualization device). In some embodiments, the inflatable device is formed of a transparent or semi-transparent material so as not to obstruct visibility of the visualization device. In some aspects, the inflatable device is clear, and filled with a clear fluid, to facilitate visualization therethrough even if in the field of view of the visualization device. In some aspects, the inflatable device is deflated to facilitate viewing. Deflation allows visualization of the extent of the separation created by the cutting device and/or inflatable device. The shape of the inflatable device may also be configured to facilitate visualization of the treatment site by a visualization device. If further separation of tissue is medically indicated, the cutting device and/or the inflatable device may be used to further separate the tissue, and visualization may once again be used to assess the extent of tissue separation, such actions being repeated as medically indicated.

In some aspects, the inflatable device is a balloon. In some aspects, the inflatable device is formed of a compliant, semi-compliant, or noncompliant material, including combinations thereof. In some aspects, the inflatable device is inflated with an inflation medium such as a fluid (e.g., saline, water, air, etc.). It will be appreciated that the inflatable device may have any of a variety of sizes, shapes, configurations, and/or dimensions, such selected in view of the nature and various other properties of the target site.

Various embodiments of tunneling systems and associated devices and methods will now be described with reference to examples illustrated in the accompanying drawings.

Reference in this specification to “one embodiment,” “an embodiment,” “some embodiments”, “other embodiments”, etc. indicates that one or more particular features, structures, concepts, and/or characteristics in accordance with principles of the present disclosure may be included in connection with the embodiment. However, such references do not necessarily mean that all embodiments include the particular features, structures, concepts, and/or characteristics, or that an embodiment includes all features, structures, concepts, and/or characteristics. Some embodiments may include one or more such features, structures, concepts, and/or characteristics, in various combinations thereof. It should be understood that one or more of the features, structures, concepts, and/or characteristics described with reference to one embodiment can be combined with one or more of the features, structures, concepts, and/or characteristics of any of the other embodiments provided herein. That is, any of the features, structures, concepts, and/or characteristics described herein can be mixed and matched to create hybrid embodiments, and such hybrid embodiment are within the scope of the present disclosure. Moreover, references to “one embodiment,” “an embodiment,” “some embodiments”, “other embodiments”, etc. in various places in the specification are not necessarily all referring to the same embodiment, nor are separate or alternative embodiments necessarily mutually exclusive of other embodiments. It should further be understood that various features, structures, concepts, and/or characteristics of disclosed embodiments are independent of and separate from one another, and may be used or present individually or in various combinations with one another to create alternative embodiments which are considered part of the present disclosure. Therefore, the present disclosure is not limited to only the embodiments specifically described herein, as it would be too cumbersome to describe all of the numerous possible combinations and subcombinations of features, structures, concepts, and/or characteristics, and the examples of embodiments disclosed herein are not intended as limiting the broader aspects of the present disclosure. It should be appreciated that various dimensions provided herein are examples and one of ordinary skill in the art can readily determine the standard deviations and appropriate ranges of acceptable variations therefrom which are covered by the present disclosure and any claims associated therewith. The following description is of illustrative examples of embodiments only, and is not intended as limiting the broader aspects of the present disclosure.

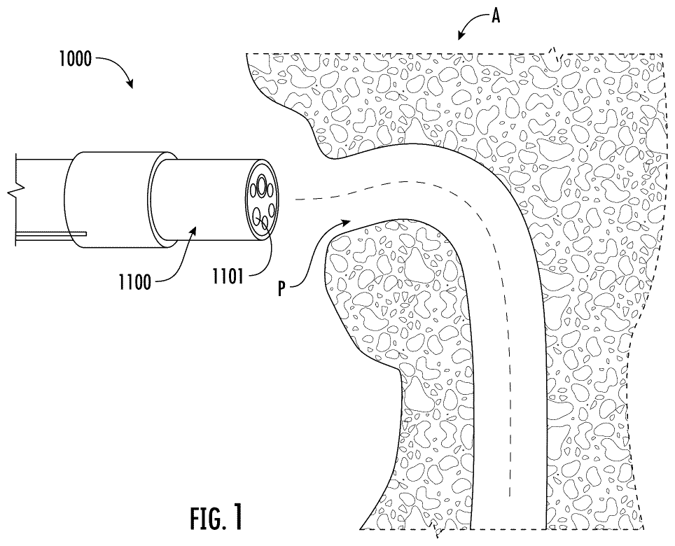

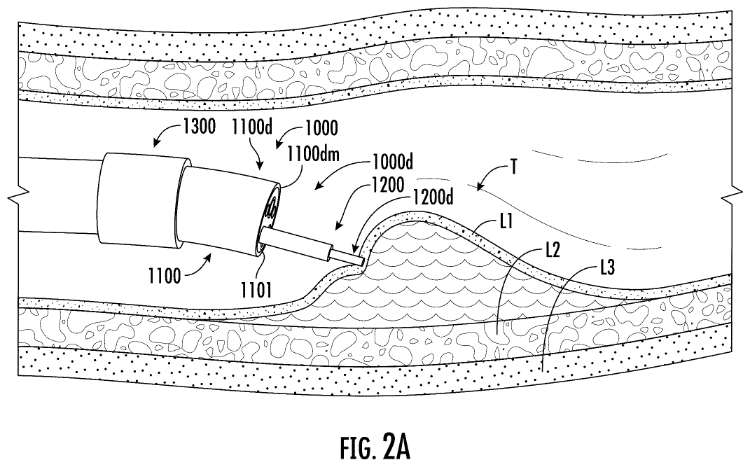

Turning now to the drawings, a system 1000 for tunneling between tissue layers is illustrated in FIG. 1 in a schematic representation of an anatomical location A within a patient. Although the illustrated anatomical location includes a natural anatomical passage P into which the system is inserted, it will be appreciated that use of a tunneling system 1000 of the present disclosure in other configurations of anatomical locations (such as within body cavities, such as within a cavity within an organ) is within the scope of the present disclosure. For instance, the anatomical passage P includes naturally-existing passages (e.g., the intestines) as well as medically-created passages (e.g., a passage created with the use of a medical instrument) or otherwise. In the schematic illustration of FIG. 1, the tunneling system 1000 is inserted into an anatomical passage P and advanced to a treatment site T at which a tunneling procedure is to be performed, as illustrated in FIG. 2A, FIG. 2B, and FIG. 2C. The illustrated example of an embodiment of a tunneling system 1000 includes an elongate delivery member 1100 configured to facilitate delivery of additional tools, instruments, device, etc. (such terms being used interchangeably herein without intent to limit) to perform a tissue tunneling procedure. In some aspects, the elongate delivery member 1100 is flexible so that it may navigate through curved or tortuous anatomical passages such as the anatomical passage P illustrated in FIG. 1. In some aspects, the elongate delivery member 1100 is tubular and defines one or more axial lumens therein so that another device of the tunneling system 1000 may be delivered through a lumen of the elongate delivery member 1100. In some embodiments, such as the embodiment illustrated in FIG. 1, FIG. 2A, FIG. 2B, and FIG. 2C, the elongate delivery member 1100 is a medical scope (e.g., an endoscope, duodenoscope, ureteroscope, bronchoscope, colonoscope, arthroscope, cystoscope, hysteroscope, etc.) with at least one lumen defining a working channel 1101 through which an additional tool of the tunneling system 1000 may be advanced/retracted. An elongate delivery member 1100 in the form of a medical scope may include other lumens therethrough, such as for irrigation, suction, insufflation, imaging devices (e.g., an optic fiber and/or camera), illuminating devices (e.g., lights such as using optic fibers and/or LED's), etc., as known to those of ordinary skill in the art.

In FIG. 2A, the distal end 1000d of the tunneling system 1000 is illustrated as positioned at the treatment site T, with a cutting device 1200 distally extended out of the working channel 1101 to cut tissue at the treatment site T. The cutting device 1200 in the illustrated example of an embodiment has a distal end 1200d is a relatively sharp device configured to create an initial incision or cut in a patient's tissue. For instance, the distal end 1200d of the cutting device 1200 may be in the form of a blade, a monopolar or bi-polar electrosurgical knife, etc. The cutting device 1200 makes the initial cut by the tunneling system 1000 into tissue at the treatment site T. As discussed in further detail below, in accordance with various principles of the present disclosure, the tunneling system 1000 further includes an inflatable device 1300 configured to perform blunt tissue dissection at the treatment site T once the cutting device 1200 makes the initial cut into the tissue, thereby facilitating separation of tissue layers, such as the layers of the tissue on opposite sides of the initial cut. Additionally or alternatively, such blunt tissue dissection may place the tissue under tension during cutting so that the user can see the separation of the tissue layers and reduce the possibility of perforation (e.g., into the peritoneum). The inflatable device 1300 may also be delivered with the elongate delivery member 1100 to the treatment site T. In some aspects, the inflatable device 1300 is mounted over the exterior of and carried by the elongate delivery member 1100 (e.g., is generally toroidal with the elongate delivery member 1100 extending therethrough), as discussed in further detail below.

In some aspects, a fluid (e.g., water, saline, air, etc.) is injected below the surface or upper layer of tissue L1 (e.g., mucosa) at the treatment site T to elevate the tissue (e.g., to form a bleb, such as illustrated in FIG. 2A) to be cut at the treatment site T to facilitate cutting of the tissue. In some aspects, the tunneling system 1000 includes an injection device (e.g., an elongate needle, such as known to those of ordinary skill in the art and thus not illustrated) which may be used to inject the fluid. In some aspects, the injection device is advanced through the working channel 1101 of the elongate delivery member 1100 to inject fluid into tissue at the treatment site T, and then retracted and removed out the proximal end of the elongate delivery member 1100.

An instrument such as a cutting device 1200 and/or injection device may be inserted and/or removed through a port at a proximal end of the elongate delivery member 1100. In some embodiments, such as embodiments in which the elongate delivery member 1100 is a medical scope, a handle is provided at the proximal end of the elongate delivery member 1100 such as to provide an access port to access at least one, and optionally each, of the one or more lumens through the elongate delivery member 1100. In some embodiments, an access port facilitates coupling of a lumen extending through the elongate delivery member 1100 with another medium or device, such as inflation/insufflation/irrigation medium(s); a vacuum source; a power source such as for an illuminating and/or visualization device. Alternatively or additionally, the handle may include one or more navigation controls (e.g., knobs, etc.) which may be used to navigate the elongate delivery member 1100, such as through tortuous anatomical passages.

Once the initial cut into tissue at the treatment site T has been made by the cutting device 1200, to separate an upper tissue layer L1 from an adjacent lower tissue layer L2, such as illustrated in FIG. 2A, the tunneling system 1000 may be further distally advanced between the tissue layers L1, L2 separated by the cut, as illustrated in FIG. 2B. The larger diameter of the elongate delivery member 1100, relative to the diameter of the cutting device 1200, may cause blunt dissection of tissue as the elongate delivery member 1100 is distally advanced between the tissue layers. In accordance with various principles of the present disclosure, to further facilitate blunt tissue dissection (beyond that which may occur upon insertion of the elongate delivery member 1100 into the cut tissue), the inflatable device 1300 is advanced and inflated between the tissue layers, such as illustrated in FIG. 2C.

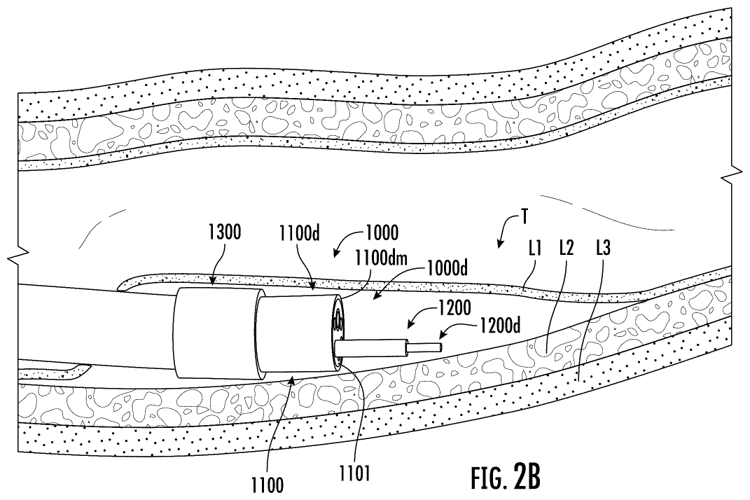

As noted above, the inflatable device 1300 may be positioned to facilitate insertion and distal advancement between the tissue layers L1, L2 separated by the cut. For instance, the inflatable device 1300 may be positioned proximal to the distalmost end 1100dm of the elongate delivery member 1100, such as illustrated in FIG. 2A. As may be appreciated with reference to FIG. 2A, FIG. 2B, FIG. 2C and FIG. 3, the inflatable device 1300 presents a wider diameter of the tunneling system 1000 than presented by the cutting device 1200. Insertion of the inflatable device 1300 into tissue cut by the cutting device 1200 thus performs blunt dissection of the cut tissue by virtue of the increased size of the tunneling system 1000 between the cut tissue. It will be appreciated that although the inflatable device 1300 is illustrated in FIG. 2A and FIG. 2B as somewhat spaced apart proximally from the distalmost end 1100dm of the elongate delivery member 1100, the inflatable device 1300 may be positioned closer to the distalmost end 1100dm, such as substantially aligned with the distalmost end 1100dm of the elongate delivery member 1100, such as in the example of an embodiment illustrated in FIG. 3.

Alternatively or additionally, the size, shape, configuration, and/or dimensions of the inflatable device 1300 may facilitate insertion thereof into tissue initially cut by a relatively sharp cutting device 1200 to perform blunt dissection of the tissue by the inflatable device 1300. For instance, the distal end 1300d of the inflatable device 1300 may be tapered or wedge-shaped or otherwise configured to gradually increase in diameter from the distalmost end 1300dm in a direction towards the proximal end 1300p of the inflatable device 1300, such as schematically illustrated in FIG. 2C and FIG. 3 when the inflatable device 1300 is inflated. It will be appreciated that the distal end 1000d of the inflatable device 1300 may be configured to gradually increase in diameter in a proximal direction even when in an uninflated configuration. In some embodiments, the proximal end 1300p of the inflatable device 1300 may exhibit a taper in a proximal direction (from the exterior of the inflatable device 1300 to the exterior of the elongate delivery member 1100), whether in an inflated or an uninflated configuration.

Alternatively or additionally, components of the tunneling system 1000 associated with the inflatable device 1300 may contribute to the size, shape, configuration, and/or dimensions of the inflatable device 1300 which facilitate insertion into cut tissue for blunt dissection of the tissue. For instance, as illustrated in FIG. 3, the tunneling system 1000 may include an adaptor 1400 positioned with respect to the inflatable device 1300 to create a gradual increase in the shape/outer diameter of the elongate delivery member 1100 in a distal to proximal direction. In some embodiments, the adaptor 1400 is configured to impart a taper from the outer diameter (e.g., from the exterior, outer surface) of the inflatable device 1300 (either in the uninflated or the inflated configuration) to the smaller outer diameter (e.g., to the exterior, outer surface) of the elongate delivery member 1100, over which the inflatable device 1300 is delivered to a treatment site T. More particularly, the example of an embodiment of an adaptor 1400 illustrated in FIG. 3 has a distal end 1400d which tapers from a location adjacent the distal end 1300d of the inflatable device 1300 towards the distal end 1000d of the tunneling system 1000 to a distal end 1100d of the elongate delivery member 1100. Optionally, the example of an embodiment of an adaptor 1400 illustrated in FIG. 3 has a proximal end 1400p which tapers from a location adjacent the proximal end 1300p of the inflatable device 1300 towards the proximal end 1000p of the tunneling system 1000 and to a section of the elongate delivery member 1100 which is proximal to the proximal end 1300p of the inflatable device 1300. The overall configuration of the combined inflatable device 1300 and adaptor 1400 facilitates insertion of the inflatable device 1300 into cut tissue by applying (optionally gradually) increasing forces to the surrounding tissue to further separate tissues held together as the inflatable device 1300 is distally advanced into the tissue to separate the tissue as desired.

As noted above, the inflatable device 1300 may be delivered with/by the elongate delivery member 1100, and/or may otherwise be associated with the elongate delivery member 1100 (e.g., mounted over the elongate delivery member 1100). In some aspects, the adaptor 1400 maintains the position of the inflatable device 1300 with respect to the elongate delivery member 1100. For instance, the adaptor 1400 may hold the proximal end 1300p and/or the distal end 1300d of the inflatable device 1300 with respect to the elongate delivery member 1100. In some aspects, the adaptor 1400 is compliant to fit on a variety of elongate delivery members 1100 having different outer diameters. For instance, the adaptor 1400 may be formed of a flexible/compliant material such as plastic and/or silicone so that the adaptor 1400 can be fitted over a variety of medical scopes, such as a variety of differently sized medical scopes (e.g., different outer diameters). In some aspects, the adaptor 1400 may be positioned over the entire length of the inflatable device 1300, or at least an intermediate portion of the inflatable device 1300 between the proximal end 1300p and distal end 1300d of the inflatable device 1300. In some embodiments, the adaptor 1400 is a single piece element positioned over the inflatable device 1300 to impart a taper such as described above. In some embodiments, the adaptor 1400 is formed from two separate elements, such as a distal adaptor 1400a and a proximal adaptor 1400b illustrated schematically in FIG. 3 (although it will be appreciated that FIG. 3 may be understood to adequately illustrate, at least schematically, a single-piece adaptor 1400 for one of ordinary skill in the art to understand such configuration of an adaptor 1400).

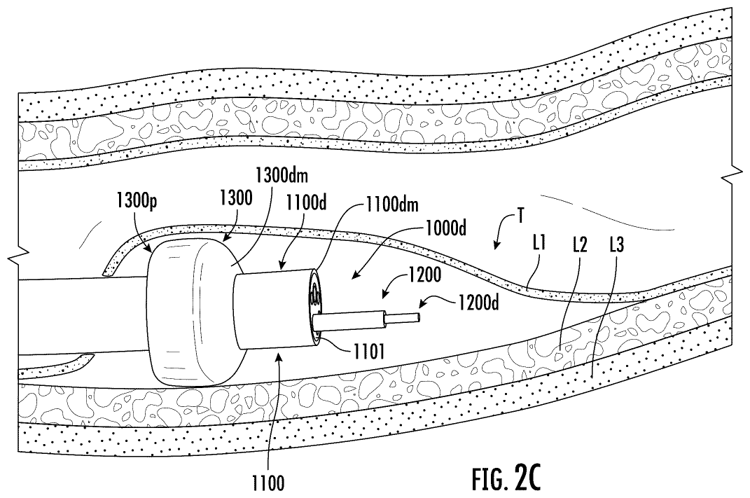

As may be appreciated, an inflatable device 1300 formed in accordance with various principles of the present disclosure may be selectively inflated and deflated, such as by fluidly coupling the inflatable device 1300 with an inflation medium (e.g., from a fluid source) and/or a drain or vacuum source. Typically, the inflation medium and/or drain/vacuum source are provided at a proximal end 1000p of the tunneling system 1000 and may be in any form such as known to those of ordinary skill in the art. As illustrated in FIG. 3 and FIG. 4, an inflation lumen 1310 extends distally from a proximal end 1000p (not shown in detail) of the tunneling system 1000 (where it is fluidly coupled with an inflation/vacuum source) to the inflatable device 1300. In some embodiments, the distal end 1310d of the inflation lumen 1310 is fluidly coupled with a proximal end 1300p of the inflatable device 1300 to be fluidly coupled with an inflation chamber 1301 formed within the inflatable device 1300, such as illustrated in FIG. 4, showing a cross-sectional view along line IV-IV in FIG. 3. It will be appreciated that although the elongate delivery member 1100 is illustrated in FIG. 4 as having a single lumen defined therein, such depiction is a schematic, simplified example, and other configurations are within the scope and spirit of the present disclosure, the present disclosure not being limited in this regard.

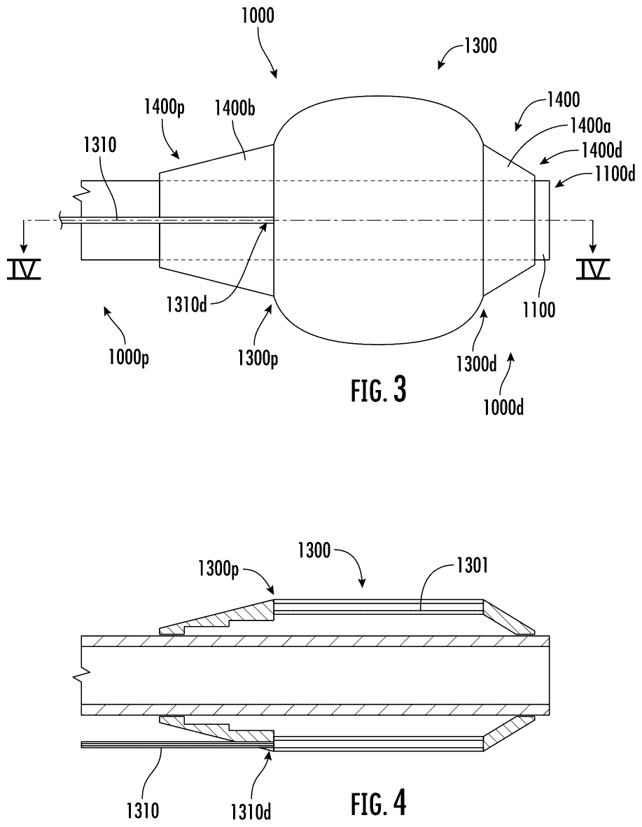

In accordance with various principles of the present disclosure, in some aspects, the position of an inflatable device 1300 of a tunneling system 1000 formed in accordance with various principles of the present disclosure may be adjustable with respect to the elongate delivery member 1100 with which the inflatable device 1300 may be delivered to a treatment site T. For instance, in the example of an embodiment illustrated in FIG. 5A, the inflatable device 1300 of the illustrated tunneling system 1000 is mounted on a sheath 1500 (e.g., over a distal end 1500d thereof). The sheath is axially translatable over the elongate delivery member 1100. For example, the sheath 1500 may have a lumen therethrough sized to slidably receive the elongate delivery member 1100 such that the sheath is distally advanceable and/or proximally retractable over and with respect to the elongate delivery member 1100 to adjust the position of the inflatable device 1300 with respect to the distal end 1100d of the elongate delivery member 1100. As illustrated in FIG. 5A, the inflatable device 1300 may be positioned adjacent the distal end 1100d of the elongate delivery member 1100 such as to be used for blunt tissue dissection. The sheath 1500 is movable with respect to the elongate delivery member 1100 so that the inflatable device 1300 may be positioned spaced proximally from the distal end 1100d of the elongate delivery member 1100, such as illustrated in FIG. 5B, such as during delivery and/or after blunt tissue dissection has been completed. As such, the use of a sheath 1500 allows movement of the inflatable device 1300 independently of the elongate delivery member 1100 to perform blunt tissue dissection without being limited by the presence of the elongate delivery member 1100.

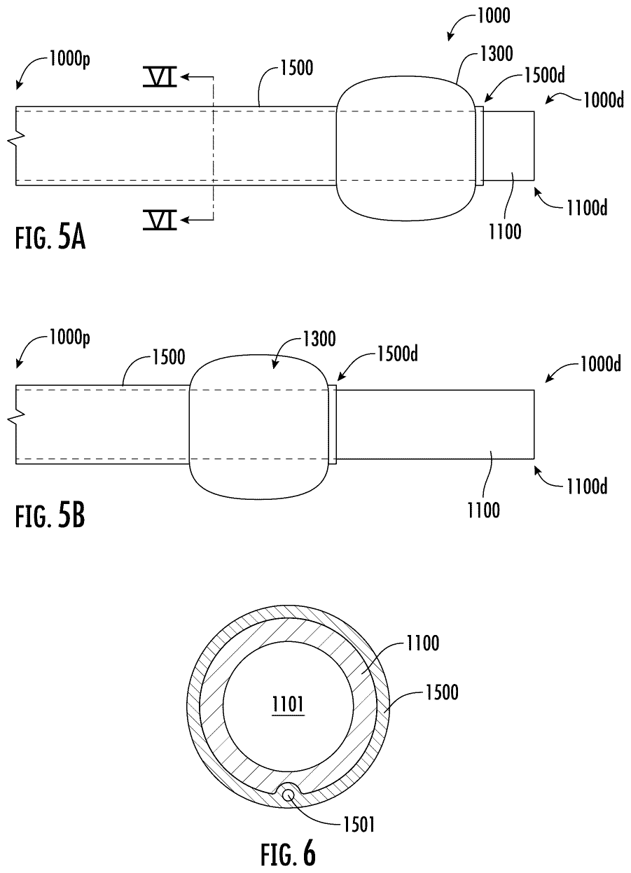

The sheath 1500 may include an inflation lumen 1501 such as illustrated in FIG. 6, showing a cross-sectional view along line VI-VI in FIG. 5A. In some aspects, the inflation lumen 1501 is positioned in a longitudinal section of the sheath 1500 with a radial thickness greater than the remaining thickness of the sheath 1500. As such, the full circumference of the sheath 1500 need not have a thickness to accommodate the inflation lumen 1501, and only a limited circumferential extent of the sheath 1500 has an increased thickness to accommodate the inflation lumen 1501. In some embodiments, the inflation lumen 1501 is radially-inwardly positioned so that the outer diameter of the sheath 1500 remains substantially constant. The region of the sheath 1500 accommodating the inflation lumen 1501 may extend radially inwardly, and may be considered to form an axial rib which may be used for alignment purposes (i.e., to mate with a corresponding axial element, such as to fit within an axial groove, extending along the exterior of the elongate delivery member 1100). Alternatively, the region of the sheath 1500 accommodating the inflation lumen 1501 may extend radially outwardly, or the sheath 1500 may have a substantially constant wall thickness circumferentially, without an increased thickness in the region of the inflation lumen 1501.

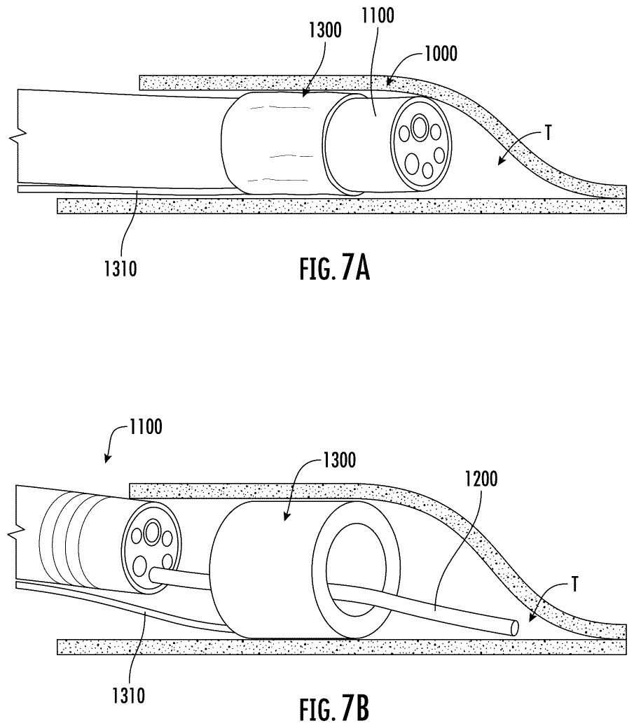

In some aspects, the inflatable device 1300 is not only movable independently of the elongate delivery member 1100, it may also be left in place between tissue to be separated by the inflatable device 1300, with the elongate delivery member 1100 proximally retracted from the treatment site T. For instance, an example of an embodiment of an inflatable device 1300 is illustrated in FIG. 7A as being delivered to a treatment site T with an elongate delivery member 1100. It will be appreciated that the axial position of the inflatable device 1300 with respect to the elongate delivery member 1100 need not be exactly as depicted in FIG. 7A, and, instead, may be closer to the distal end 1100d of the elongate delivery member 1100. Once the inflatable device 1300 is delivered to the treatment site T and inflated, the elongate delivery member 1100 may be proximally retracted, leaving the elongate delivery member 1100 in place, unsupported by the elongate delivery member 1100, such as illustrated and FIG. 7B. To retrieve the inflatable device 1300 (such as to remove the inflatable device 1300 from the treatment site T), the elongate delivery member 1100 may be distally advanced to its initial position extending through the toroidal inflatable device 1300 and the inflatable device 1300 may be deflated (partially or fully) to engage the exterior of the elongate delivery member 1100 to be proximally withdrawn from the treatment site T with the elongate delivery member 1100. As noted above, an inflatable device 1300 of the present disclosure may be formed of a compliant or semi-compliant or noncompliant material. In some aspects, the material of the inflatable device 1300 may be such that when the inflatable device 1300 is deflated/partially deflated, the inflatable device 1300 has a radially-inward contraction force which maintains the inflatable device 1300 on the exterior of the delivery member 1100.

It is to be understood by one of ordinary skill in the art that apparatuses and methods discussed herein are examples of apparatuses and/or methods implemented in accordance with one or more principles of this disclosure. These examples are not the only way to implement these principles but are merely examples, not intended as limiting the broader aspects of the present disclosure. Thus, references to elements or structures or features in the drawings must be appreciated as references to examples of embodiments of the disclosure, and should not be understood as limiting the disclosure to the specific elements, structures, or features illustrated. It will be appreciated features described with respect to one embodiment typically may be applied to another embodiment, whether or not explicitly indicated. The various features hereinafter described may be used singly or in any combination thereof. Therefore, the present invention is not limited to only the embodiments specifically described herein. It is to be understood by one of ordinary skill in the art that the present discussion is a description of illustrative examples of embodiments only, and is not intended as limiting the broader aspects of the present disclosure.

The foregoing discussion has broad application and has been presented for purposes of illustration and description and is not intended to limit the disclosure to the form or forms disclosed herein. It will be understood that various additions, modifications, and substitutions may be made to embodiments disclosed herein without departing from the concept, spirit, and scope of the present disclosure. In particular, it will be clear to those skilled in the art that principles of the present disclosure may be embodied in other forms, structures, arrangements, proportions, and with other elements, materials, and components, without departing from the concept, spirit, or scope, or characteristics thereof. For example, various features of the disclosure are grouped together in one or more aspects, embodiments, or configurations for the purpose of streamlining the disclosure. However, it should be understood that various features of the certain aspects, embodiments, or configurations of the disclosure may be combined in alternate aspects, embodiments, or configurations. While the disclosure is presented in terms of embodiments, it should be appreciated that the various separate features of the present subject matter need not all be present in order to achieve at least some of the desired characteristics and/or benefits of the present subject matter or such individual features. One skilled in the art will appreciate that the disclosure may be used with many modifications or modifications of structure, arrangement, proportions, materials, components, and otherwise, used in the practice of the disclosure, which are particularly adapted to specific environments and operative requirements without departing from the principles or spirit or scope of the present disclosure. For example, elements shown as integrally formed may be constructed of multiple parts or elements shown as multiple parts may be integrally formed, the operation of elements may be reversed or otherwise varied, the size or dimensions of the elements may be varied. Similarly, while operations or actions or procedures are described in a particular order, this should not be understood as requiring such particular order, or that all operations or actions or procedures are to be performed, to achieve desirable results. Additionally, other implementations are within the scope of the following claims. In some cases, the actions recited in the claims can be performed in a different order and still achieve desirable results. The presently disclosed embodiments are therefore to be considered in all respects as illustrative and not restrictive, the scope of the claimed subject matter being indicated by the appended claims, and not limited to the foregoing description or particular embodiments or arrangements described or illustrated herein. In view of the foregoing, individual features of any embodiment may be used and can be claimed separately or in combination with features of that embodiment or any other embodiment, the scope of the subject matter being indicated by the appended claims, and not limited to the foregoing description.

In the foregoing description and the following claims, the following will be appreciated. The phrases “at least one”, “one or more”, and “and/or”, as used herein, are open-ended expressions that are both conjunctive and disjunctive in operation. The terms “a”, “an”, “the”, “first”, “second”, etc., do not preclude a plurality. For example, the term “a” or “an” entity, as used herein, refers to one or more of that entity. As such, the terms “a” (or “an”), “one or more” and “at least one” can be used interchangeably herein. As used in this specification and the appended claims, the term “or” is generally employed in its sense including “and/or” unless the content clearly dictates otherwise. As used herein, the conjunction “and” includes each of the structures, components, features, or the like, which are so conjoined, unless the context clearly indicates otherwise, and the conjunction “or” includes one or the others of the structures, components, features, or the like, which are so conjoined, singly and in any combination and number, unless the context clearly indicates otherwise. All directional references (e.g., proximal, distal, upper, lower, upward, downward, left, right, lateral, longitudinal, front, back, top, bottom, above, below, vertical, horizontal, radial, axial, clockwise, counterclockwise, and/or the like) are only used for identification purposes to aid the reader's understanding of the present disclosure, and/or serve to distinguish regions of the associated elements from one another, and do not limit the associated element, particularly as to the position, orientation, or use of this disclosure. Connection references (e.g., attached, coupled, connected, engaged, joined, etc.) are to be construed broadly and may include intermediate members between a collection of elements and relative movement between elements unless otherwise indicated. As such, connection references do not necessarily infer that two elements are directly connected and in fixed relation to each other. Identification references (e.g., primary, secondary, first, second, third, fourth, etc.) are not intended to connote importance or priority, but are used to distinguish one feature from another.

The following claims are hereby incorporated into this Detailed Description by this reference, with each claim standing on its own as a separate embodiment of the present disclosure. In the claims, the terms “comprises”, “comprising”, “includes”, and “including” do not exclude the presence of other elements, components, features, groups, regions, integers, steps, operations, etc. Additionally, although individual features may be included in different claims, these may possibly advantageously be combined, and the inclusion in different claims does not imply that a combination of features is not feasible and/or advantageous. In addition, singular references do not exclude a plurality. Reference signs in the claims are provided merely as a clarifying example and shall not be construed as limiting the scope of the claims in any way.

What is claimed is:

1. A system for tunneling between tissue layers, said system comprising:
an elongate delivery member;
a cutting device deliverable with said elongate delivery member; and
an inflatable device positionable on a distal end of said elongate delivery member, wherein said inflatable device is shaped to facilitate insertion into a cut area of tissue cut by said cutting device.

2. The system of claim 1, wherein said elongate delivery member is tubular and defines a delivery lumen therethrough.

3. The system of claim 2, wherein said cutting device is deliverable through said delivery lumen of said elongate delivery member.

4. The system of claim 3, wherein said inflatable device is deliverable over the exterior of said elongate delivery member.

5. The system of claim 4, wherein said elongate delivery member is a medical scope, and said delivery lumen is the working channel of said medical scope.

6. The system of claim 1, wherein said inflatable device is deliverable over the exterior of said elongate delivery member.

7. The system of claim 6, wherein said inflatable device is axially translatable distally and proximally over the exterior of said elongate delivery member.

8. The system of claim 7, wherein said inflatable device is mounted over a sheath and said sheath is axially translatable distally and proximally over said elongate delivery member.

9. The system of claim 7, wherein said inflatable device is movable distally off the exterior of said elongate delivery member.

10. The system of claim 6, further comprising an adaptor configured to maintain said inflatable device with respect to said elongate delivery member.

11. The system of claim 10, wherein said adaptor has a distal end tapered from a position adjacent a distal end of said inflatable device distally to the exterior of said elongate delivery member.

12. The system of claim 10, wherein said adaptor is formed of a compliant material capable of fitting over elongate delivery members having different outer diameters.

13. The system of claim 6, wherein said inflatable device is tapered in a distal direction from an exterior thereof to an exterior of said elongate delivery member.

14. The system of claim 3, wherein said inflatable device is tapered in a proximal direction from an exterior thereof to an exterior of said elongate delivery member.

15. The system of claim 1, wherein said cutting device is a sharp cutting device selected from the group consisting of: a blade, a knife, or an electrosurgical knife.

16. A method of performing blunt dissection of tissue, said method comprising:
advancing a cutting device to a target site within a patient;
cutting tissue with the cutting device;
advancing an inflatable device into the cut tissue; and
inflating the inflatable device to separate the cut tissue further.

17. The method of claim 16, further comprising advancing the cutting device into the mucosa of the patient to separate the mucosa from the submucosa.

18. The method of claim 16, further comprising advancing the cutting device through a lumen of an elongate delivery member and advancing the inflatable member over the exterior of the elongate delivery member.

19. A method of performing endoscopic tissue dissection, said method comprising:
advancing a cutting device through a working channel of a medical scope to a target site within a patient;
cutting tissue with the cutting device;
advancing an inflatable device over the medical scope and into the cut tissue; and
inflating the inflatable device to separate the cut tissue further.

20. The method of claim 19. further comprising advancing the cutting device into the mucosa of the patient to separate the mucosa from the submucosa.
